SEQUENCE LISTING (10 sequences)

SEQ ID NO: 1 - protein, 7 aa, Artificial Sequence
  Synthetic peptide
TTQGNPQ

SEQ ID NO: 2 - protein, 7 aa, Artificial Sequence
  Synthetic peptide
YEQHHPG

SEQ ID NO: 3 - protein, 7 aa, Artificial Sequence
  Synthetic peptide
TTPHAWL

SEQ ID NO: 4 - protein, 7 aa, Artificial Sequence
  Synthetic peptide
TDNTAKN

SEQ ID NO: 5 - protein, 7 aa, Artificial Sequence
  Synthetic peptide
KIGFHGK

SEQ ID NO: 6 - protein, 7 aa, Artificial Sequence
  Synthetic peptide
KTHAQHE

SEQ ID NO: 7 - protein, 7 aa, Artificial Sequence
  Synthetic peptide
LSEQNRS

SEQ ID NO: 8 - protein, 7 aa, Artificial Sequence
  Synthetic peptide
PMPRPSS

SEQ ID NO: 9 - protein, 7 aa, Artificial Sequence
  Synthetic peptide
SLTTSTL

SEQ ID NO: 10 - protein, 7 aa, Artificial Sequence
  Synthetic peptide
ATTKFSG

(19) United States
(12) Patent Application Publication
(10) Pub. No.: US 2012/0269882 A1
(43) Pub. Date: Oct. 25, 2012

(54) Brain Delivery of Insulin to Treat Systemic Inflammation

(75) Inventor: Christoph Buettner, New York, NY (US)

(73) Assignee: MOUNT SINAI SCHOOL OF MEDICINE Office of Technology & Business Development, New York, NY (US)

(21) Appl. No.: 13/321,471

(22) PCT Filed: May 19, 2010
(86) PCT No.: PCT/US10/35459

§ 371 Date: Jul. 12, 2012

Related U.S. Application Data
(60) Provisional application No. 61/179,624, filed on May 19, 2009.

(51) Int. Cl. A61K 38/28; A61M 25/00; C07K 19/00; A61M 31/00; A61P 29/00; A61K 9/127

(52) U.S. Cl. 424/450; 514/5.9; 514/6.5; 530/303; 604/93.01; 604/264

(57) ABSTRACT

Methods and composition for the treatment of systemic inflammation, such as bacterial or viral sepsis, are described. Methods of treating systemic inflammation comprise delivery of insulin to the brain, for example by intracranial or intranasal administration. Insulin delivery systems and brain-targeted insulin compositions and polypeptides are also provided.

This present application claims the benefit of priority U.S. Provisional Patent Application Ser. No. 61/179,624 filed May 19, 2009, which is incorporated herein by reference in its entirety.

This invention was made with U.S. government support under grant no. KO8 DK074873, awarded by the National Institute of Diabetes and Digestive and Kidney Diseases of the National Institutes of Health. The U.S. government has certain rights in the invention.

### FIELD OF THE INVENTION

The present invention relates to methods and compositions for the treatment of systemic inflammation by delivery of insulin to the brain.

### BACKGROUND OF THE INVENTION

Critically ill patients are not only at high risk of death, but often develop what is commonly referred to as “stress diabetes.” This “diabetes of injury” is characterized by hyperglycemia and insulin resistance. Moreover, patients with type 2 diabetes mellitus (T2DM) have a poorer prognosis than non-diabetic patients in the setting of critical illness. Critical illness thus induces T2DM, and T2DM worsens critical illness. A recent landmark study (Van den Berghe, N. Engl. J. Med., 2001) showed that intensive insulin therapy in critically ill patients significantly increases survival and reduces morbidity. However, follow up studies demonstrated a high risk for hypoglycemia, severely limiting the beneficial effects of intensive insulin therapy for critically ill patients. Thus, there remains a need for insulin therapies that can be used to effectively treat inflammatory conditions such as sepsis without triggering hypoglycemia in treated patients.

### SUMMARY

In a first embodiment, there is provided a method for treating systemic inflammation in a subject comprising administering insulin to the subject wherein the insulin is delivered to the brain of the subject. For example, methods described herein may be used for the treatment of viral or bacterial sepsis or to treat systemic inflammatory response syndrome (SIRS). In certain aspects, systemic inflammation is treated by administration of insulin intracranially or intranasally. Thus, delivery of insulin to the brain can result in brain insulin concentrations that are higher than serum insulin concentrations thereby allowing a dosage of insulin to be used which is effective to treat systemic inflammation while not causing serum hypoglycemia in the subject. For example, in certain aspects, the dose of insulin administered to the subject is defined as a dose effective to treat systemic inflammation and that does not result in hypoglycemia in the subject. Exemplary dosages include dosages of insulin up to about 20 microUnits, dosages up to about 100 Units, dosages of 20 microUnits to 100 Units and a dosage of 20 microUnits. A range of dosage concentrations may be used, along with a range of delivery times. Thus, for example, a preferred administration would be the delivery of 5 microUnits per hour for 4 hours, yielding an administration of 20 microUnits.

In some cases, a subject having systemic inflammation is defined as the human subject. In certain aspects, it is contemplated that the subject is unconscious (e.g., a subject in a hospital intensive care unit). Moreover, in certain aspects, methods described herein are used to treat viral or bacterial sepsis. For example, bacterial sepsis can be Salmonella, Staphylococcus, Streptococcus, Enterococcus, Escherichia, Klebsiella, Enterobacter, Pseudomonas, Yersinia, Campylobacter, Bacillus, Treponema or Fusobacterium sepsis. In further aspects, methods described herein comprise administering a second therapeutic to the subject, such as, for example, an antibiotic, an anti-inflammatory and/or an antiviral composition. In particular aspects, a second therapeutic is administered via the same route of administration as the insulin composition or is administered via a different route of administration such as intravenously or orally. Thus, in certain aspects, an insulin composition for intranasal or intracranial administration further comprises a second therapeutic such as an antibiotic or antiviral.

In some aspects, methods disclosed herein concern administration of insulin to a subject such that the insulin is delivered to the brain of the subject. For instance, insulin can be administered intracranially or intranasally and, in various aspects, is administered 1, 2, 3, 4, 5 or more times. Intracranial administration of insulin is accomplished, in some aspects, by providing the insulin though a cannula. In certain specific aspects, insulin is delivered intracranially to the third ventricle of the subject. In various aspects, a variety of methods is used to deliver insulin intranasally. In some aspects, the insulin is lyophilized and delivered as a dispersed powder formulation. In certain other aspects, insulin is provided in a solution (e.g., saline) formulated for intranasal administration. Moreover, in some cases, intranasal administration of insulin is accomplished by applying pressure to an insulin composition. Thus, in some cases, an insulin composition for intranasal delivery is comprised in a syringe, a nebulizer, a respirator or a squeeze bottle such that pressure can be applied to facilitate insulin delivery to the intranasal passages of the subject (e.g., via a mechanical action by a human or pump or via a compressed gas).

Insulin is available in a variety of formulations any of which may be used for methods disclosed herein. In some cases, insulin is formulated to enhance uptake to the brain from the intranasal passages. For example, insulin may be formulated with liposomes or microspheres suitable for intranasal delivery. Microspheres, in some cases, are composed of polysaccharides, proteins (e.g., gelatin, albumin, or collagen), or synthetic polymers (e.g., starch, dextran, hyaluronic acid, gellan gum and pectin). A detailed description of microspheres for use in accordance with the instant disclosure is found in U.S. Pat. No. 5,707,644, incorporated herein by reference.

In certain aspects, the insulin is defined as recombinant insulin. Moreover, in certain aspects, the insulin may be modified to enhance stability, uptake or targeting of the insulin to the brain. For example, insulin can be PEGylated or fused with an antibody constant domain to enhance stability. Moreover, in some cases, insulin is fused with or conjugated to a monoclonal antibody or antibody fragment that enhances insulin uptake or targeting to the brain. In some aspects, a recombinant insulin peptide is fused to a peptide that enhances brain targeting of the insulin to the brain. Peptide sequences that enhance brain targeting have been described, for example by Wan et al., Peptides, 30:343-350, 2009, incorporated herein by reference. Thus, in certain aspects, insulin is fused to a peptide that enhances uptake to the brain selected from the group consisting of peptides comprising the sequence TTQGNPQ (SEQ ID NO:1), YEQHHPG (SEQ ID NO:2), TTPHAWL (SEQ ID NO:3), TDNTAKN (SEQ ID NO:4), KIGFHGK (SEQ ID NO:5), KTHAQHE (SEQ ID NO:6), LSEQNRS (SEQ ID NO:7), PMPRPSS (SEQ ID NO:8), SLTTSTL (SEQ ID NO:9) and ATTKFSG (SEQ ID NO:10). In still further aspects, insulin is fused to a peptide having a sequence TTPHAWL (SEQ ID NO: 3). The skilled artisan recognizes that insulin, in particular aspects, is fused to a peptide at either the amino or carboxyl terminus and, optionally, comprises a linker sequence between the targeting peptide and insulin. Thus, in one aspect, the disclosure provides a polypeptide comprising a human insulin amino acid sequence, or insulin peptide, fused to a targeting peptide that enhances insulin uptake to the brain.

In still further aspects, pharmaceutical compositions comprising insulin are provided. For example, in one aspect, the disclosure provides an insulin composition for intracranial administration comprising insulin formulated in artificial cerebrospinal fluid (ACSF). Formulations for ACSF are known in the art and certain specific formulations are detailed herein. Alternatively, there is provided, a pharmaceutical composition for intranasal administration comprising a dosage of insulin effective to treat systemic inflammation when administered to a subject via the intranasal route in a carrier formulated for intranasal administration. For example, a carrier for intranasal administration, in some aspects, comprises a saline solution and an agent which enhances insulin uptake or targeting to the brain, such as a liposome or microsphere. Moreover, in certain aspects, insulin compositions are administered by a syringe, a nebulizer, a respirator (or a cartridge that is a designed for coupling to a nebulizer or respirator) or a squeeze bottle to facilitate intranasal administration. In still further aspects, insulin compositions for intracranial or intranasal administration can comprise a second therapeutic agent such as, for example, an antiviral, an antibiotic or an anti-inflammatory agent.

In yet further embodiments, insulin delivery systems are provided. For example, in certain aspects, the disclosure provides an intracranial insulin delivery system comprising insulin formulated for intracranial delivery and a brain infusion cannula. Intracranial insulin delivery systems can optionally include an infusion pump that can be coupled to the brain infusion cannula, spacer joints which allow adjustments of the depth of cannula, instructions for the use of the system and/or compositions comprising additional therapeutics for intracranial administration. Alternatively, the disclosure provides an intranasal insulin delivery system comprising insulin formulated for intranasal delivery and a pressure source sufficient to deliver the insulin to the intranasal passages of a subject. In certain aspects, the pressure source is defined as supplying sufficient pressure to deliver an insulin composition to the intranasal passages of an unconscious subject. For example, the pressure source may be a syringe, a nebulizer, a respirator a squeeze bottle or a pump. In certain aspects, an insulin delivery system comprises a single unit dosage of insulin effective for the treatment of systemic inflammation in a human subject. Thus, in still further aspects, there is provided a kit for the treatment of systemic inflammation, such as sepsis, comprising one or more unit doses of insulin formulated for intranasal administration and one or more antibiotic or antiviral agents, wherein said doses of insulin are effective to treat sepsis.

In additional embodiments, the disclosure provides uses of compositions described herein for the preparation of medicaments. Other related aspects are also provided in the instant invention.

The foregoing summary is not intended to define every aspect of the invention, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE DRAWING

The following drawings form part of the present specification and are included to further illustrate aspects of the present invention. The invention may be better understood by reference to the drawings in combination with the detailed description of the specific embodiments presented herein.

FIG. 1A-B: FIG. 1A, Experimental protocol for high fat diet feeding and intracerebroventricular (ICV) cannula implantation. 4 week old male C57B1/6 mice were fed a high fat diet for 4 weeks and ICV cannulae were implanted. After 5 days recovery from surgery infusion studies were performed. FIG. 1B, Protocol for infusion studies. Mice were injected with lipopolysaccharides (LPS) (1 mg/kg, i.p.) and randomly assigned to receive ICV insulin or vehicle infusions for 4 hours until necropsy.

FIG. 2: Diagram indicating placement of ICV cannula targeting the third ventricle. Position is 0.38 mm anteroposterior and 5 mm below bregma.

FIG. 3: ICV insulin treatment reduces LPS induced pro-inflammatory cytokine levels. Mice were injected with LPS (1mg/kg, i.p.) infused with central insulin (0.02 mU/h) or vehicle for 4 h and sacrificed. Serum concentrations of TNF-α and IL-6 were determined by ELISA. n=12-14 per group, *p<0.05.

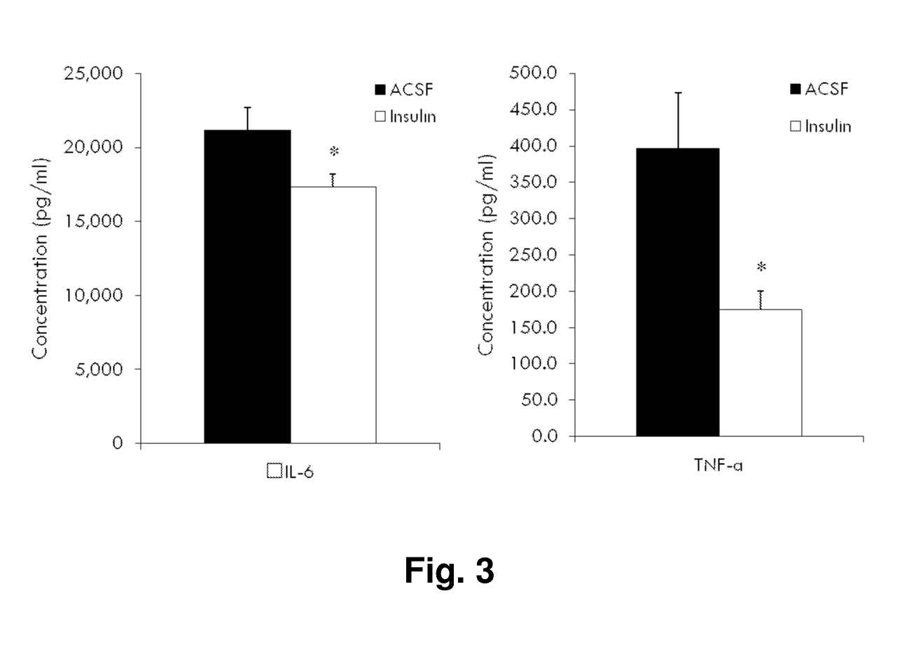

FIG. 4: ICV insulin treatment reduces LPS induced pro-inflammatory proteins in spleen. Quantification of Western blot analyses from spleen tissue lysates expressed as fold change to vehicle and normalized over β-actin. Activation of the transcription factors Stat1 and Stat3 is decreased. The pro-inflammatory markers IL-1β, COX2 and IKK-β are reduced in the ICV insulin infused animals. n=6 per group, *p<0.05.

FIG. 5: ICV insulin treatment reduces LPS induced pro-inflammatory proteins in lung. Quantification of Western blot analyses from lung tissue lysates expressed as fold change to vehicle and normalized over α-tubulin. Activation of the transcription factors Stat1 is decreased by central insulin. The level of the pro-inflammatory cytokine IL-1β is reduced in the ICV insulin infused animals. n=6 per group, *p<0.05.

FIG. 6: ICV insulin treatment reduces LPS induced pro-inflammatory proteins in liver. Quantification of western blot analyses from liver tissue lysates expressed as fold change to vehicle and normalized over β-actin. Activation of the transcription factors Stat1 and Stat3 is decreased. The pro-inflammatory cytokine IL-1β is reduced in the ICV insulin infused animals. n=6 per group, *p<0.05.

FIG. 7: ICV insulin induced mRNA levels of pro-inflammatory genes in rat liver. Fold change in mRNA levels of the pro-inflammatory genes TNF-α, IL-6, MIP-2 and IP10 and anti-inflammatory gene SRC3 after LPS injection and treatment with ICV insulin for 4 h, in liver tissue as determined by real-time PCR. mRNA levels of each gene were normalized to their respective 18S RNA levels. n=3-6 per group, *p<0.05.

FIG. 8: ICV glucose infusion increases LPS-induced pro-inflammatory proteins in liver. Quantification of western blot analyses from liver tissue lysates expressed as fold change to vehicle and normalized over β-actin. Mice were injected with LPS (1 mg/kg, i.p.) and randomly assigned to receive ICV glucose (4 mM) or vehicle infusions for 4 hours until necropsy. Activation of the transcription factors Stat1 and Stat3 is increased. Higher levels of IKK-β involved in NFκB signaling and PDI, a marker for ER stress, were detectable in the ICV glucose infused animals. n=6 per group, *p<0.05.

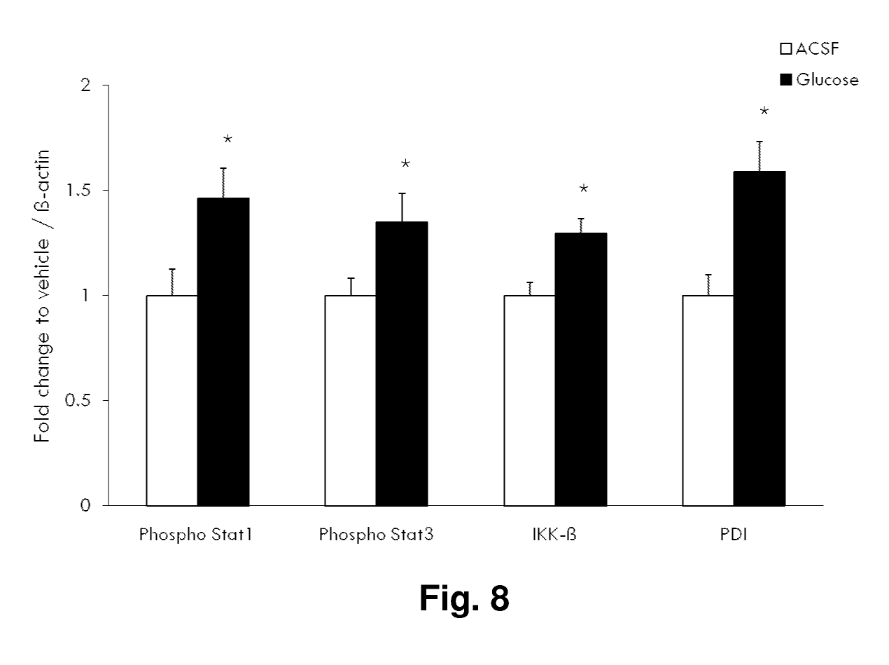

FIG. 9: ICV insulin improves survival in a mouse model of sepsis. 4 weeks old C57B16 mice were fed high fat diet for 4 weeks and ICV cannulas were implanted. Mice were injected with LPS (5 mg/kg, ip) and randomly assigned to receive ICV insulin or vehicle infusions for up to 5 days (n=11 per group). Survival was assessed daily as depicted. ICV insulin significantly (p=0.0194) improved survival.

### DETAILED DESCRIPTION

Systemic inflammation, such as inflammation caused by bacterial sepsis can result in serious clinical disease with a high rate of patient mortality. One therapeutic currently used to mitigate the effects of systemic inflammation is intravenous insulin therapy. Intensive insulin therapy in critically ill patients not only raises insulin levels but also ameliorates hyperglycemia which, recent findings indicate, has potent pro-inflammatory effects. However, intensive insulin therapy in patients carries the risk of hypoglycemia and thus has limited usefulness. Compositions and methods described herein, in various aspects, are used for brain delivery of insulin which, as demonstrated here, provides a potent anti-inflammatory effect with greatly reduced risk of inducing hypoglycemia.

One well-established model for systemic inflammation and sepsis involves LPS administration to mice. Using this model system, it has now been shown that intracranial delivery of insulin to LPS-treated mice results in potent anti-inflammatory effects. Importantly, insulin administered to the central nervous system (CNS) was able to significantly improve the survival of LPS-treated mice relative to control animals. It is recognized that insulin can be delivered to the brain by intranasal as well as intracranial routes (see, e.g., Hanson et al., BMC Neurosci., 9(Suppl. 3):S5, 2008, incorporated herein by reference). Thus, the instant disclosure provides methods and insulin compositions for the treatment of systemic inflammation by insulin administration to the CNS (e.g., via the intracranial or intranasal route).

Pharmaceutical Compositions and Formulations

Aqueous compositions of the present invention comprise an effective amount of insulin, dissolved or dispersed in a pharmaceutically acceptable carrier or aqueous medium. The phrases “pharmaceutically or pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, dispersion media, coatings, antibacterial, antiviral and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Dispersions also can be prepared in glycerol, liquid polyethylene glycols, and mixtures thereof and in oils. Under ordinary conditions of storage and use, these preparations contain a preservative to prevent the growth of microorganisms. Aqueous carriers include water, alcoholic/aqueous solutions, saline solutions, parenteral vehicles such as sodium chloride, Ringer's dextrose, etc. Preservatives include antimicrobial agents, anti-oxidants, chelating agents and inert gases. The pH and exact concentration of the various components the pharmaceutical composition are adjusted according to well known parameters. Supplementary active ingredients also can be incorporated into the compositions.

Intranasal Formulations and Systems

Insulin, in various aspects, is provided in a metered dose which, when administered intranasally, is well below the level associated with hypoglycemia. Insulin is a peptide hormone comprising 51 amino acids. The terms insulin, insulin peptide, and insulin polypeptide are used interchangeably herein. In various aspects, the disclosure includes insulin analogs, insulin variants, insulin derivatives, and fragments of insulin that have insulin biological activity. In some aspects, a dose of a second therapeutic agent (e.g., a steroid) is provided in an amount effective to alleviate inflammation.

In various aspects, insulin dosages include dosages of insulin at about 1 microUnit, at about 2 microUnits, at about 3 microUnits, at about 4 microUnits, at about 5 microUnits, at about 6 microUnits, at about 7 microUnits, at about 8 microUnits, at about 9 microUnits, at about 10 microUnits, at about 20 microUnits, at about 30 microUnits, at about 40 microUnits, at about 50 microUnits, at about 60 microUnits, at about 70 microUnits, at about 80 microUnits, at about 90 microUnits, at about 100 microUnits, at about 150 microUnits, at about 200 microUnits, at about 250 microUnits, at about 300 microUnits, at about 350 microUnits, at about 400 microUnits, at about 450 microUnits, at about 500 microUnits, at about 550 microUnits, at about 600 microUnits, at about 650 microUnits, at about 700 microUnits, at about 750 microUnits, at about 800 microUnits, at about 850 microUnits, at about 900 microUnits, at about 950 microUnits and at about 100 microUnits.

In other aspects, insulin dosages include dosages of insulin at about 1 Unit, at about 10 Units, at about 20 Units, at about 30 Units, at about 40 Units, at about 50 Units, at about 60 Units, at about 70 Units, at about 80 Units, at about 90 Units, at about 100 Units, at about 150 Units, at about 200 Units, at about 250 Units, at about 300 Units, at about 350 Units, at about 400 Units, at about 450 Units, at about 500 Units, at about 550 Units, at about 600 Units, at about 650 Units, at about 700 Units, at about 750 Units, at about 800 Units, at about 850 Units, at about 900 Units, at about 950 Units and at about 100 Units.

In other aspects, a range of insulin dosage concentrations may be used, along with a range of delivery times. Dosage ranges include, but are not limited to about 1 microUnit to about 100 Units, about 5 microUnits to about 20 microUnits, about 20 microUnits to about 100 Units and about 10 microUnits to about 100 microUnits. Thus, for example, in a certain aspect, administration would be the delivery of about 5 microUnits per hour for about 4 hours, yielding an administration of about 20 microUnits. In another aspect, the delivery times range from about several hours to about several days. Delivery times include, but are not limited to about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours and about 24 hours. In other aspects, delivery times include one day to over several days.

The disclosure provides a device for unit dose administration of insulin, wherein the device comprises a nasal inhaler containing an aerosol formulation of insulin and a pharmaceutically acceptable dispersant, wherein the device is metered to disperse an amount of the aerosol formulation that contains a dose of insulin effective to reduce inflammation or reduce at least one marker of inflammation (e.g., TNF-α). The dispersant, in certain aspects, is a surfactant, such as, but not limited to, polyoxyethylene fatty acid esters, polyoxyethylene fatty acid alcohols, and polyeoxyethylene sorbitan fatty acid esters. Phospholipid-based surfactants also may be used.

In other embodiments, the aerosol formulation of insulin is provided as a dry powder aerosol formulation in which the insulin is present as a finely divided powder. The dry powder formulation can further comprise a bulking agent, such as, but not limited to, lactose, sorbitol, sucrose and mannitol.

In another specific embodiment, the aerosol formulation is a liquid aerosol formulation further comprising a pharmaceutically acceptable diluent, such as, but not limited to, sterile water, saline, buffered saline and dextrose solution.

In still further aspects, insulin is formulated with an agent that targets the insulin to the brain and/or a “mucosal penetration enhancer,” i.e., a reagent that increases the rate or facility of transmucosal penetration of insulin, such as, but not limited to, a bile salt, fatty acid, surfactant or alcohol. In specific embodiments, the permeation enhancer is sodium cholate, sodium dodecyl sulphate, sodium deoxycholate, taurodeoxycholate, sodium glycocholate, dimethylsulfoxide or ethanol.

As used herein, the term “dispersant” refers to an agent that assists aerosolization of the insulin or absorption of the insulin in intranasal mucosal tissue, or both. In a specific aspect, the dispersant is a mucosal penetration enhancer. In various aspects, the dispersant is pharmaceutically acceptable. As used herein, the term “pharmaceutically acceptable” means approved by a regulatory agency of the Federal government, a state government or listed in the U.S. Pharmacopeia, or other generally recognized pharmacopeia for use in animals, and more particularly in humans.

Suitable dispersing agents are well known in the art, and include, but are not limited to, surfactants and the like. Such surfactants are generally used in the art to reduce surface induced aggregation of insulin caused by atomization of the solution forming the liquid aerosol and, in various aspects, are used in the methods and devices of the present invention. Examples of such surfactants include, but are not limited to, surfactants, such as polyoxyethylene fatty acid esters and alcohols, and polyoxyethylene sorbitan fatty acid esters. Amounts of surfactants used will vary, being generally within the range or 0.001 and 4% by weight of the formulation. Suitable surfactants are well known in the art and, in various aspects, are selected on the basis of desired properties, depending on the specific formulation, concentration of insulin, diluent (in a liquid formulation) or form of powder (in a dry powder formulation), and the like.

The liquid aerosol formulations contain insulin and a dispersing agent in a physiologically acceptable diluent. The dry powder aerosol formulations, in various aspects, consist of a finely divided solid form of insulin and a dispersing agent. With either the liquid or dry powder aerosol formulation, the formulation, in certain aspects, is aerosolized. That is, it can be broken down into liquid or solid particles in order to ensure that the aerosolized dose actually reaches the mucous membranes of the nasal passages or the lung. The term “aerosol particle” is used herein to describe the liquid or solid particle suitable for nasal or pulmonary administration, i.e., that will reach the mucous membranes. Other considerations, such as construction of the delivery device, additional components in the formulation, and particle characteristics are important. These aspects of nasal administration of a drug are well known in the art, and manipulation of formulations, aerosolization means and construction of a delivery device require at most routine experimentation by one of ordinary skill in the art.

With regard to construction of the delivery device, any form of aerosolization known in the art, including, but not limited to, spray bottles, nebulization, atomization or pump aerosolization of a liquid formulation, and aerosolization of a dry powder formulation, is used in the delivery of insulin.

As noted above, in some aspects, the device for aerosolization is a metered dose inhaler. A metered dose inhaler provides a specific dosage when administered, rather than a variable dose, depending on administration. In various aspects, such a metered dose inhaler is used with either a liquid or a dry powder aerosol formulation. Metered dose inhalers are well known in the art.

For nasal administration, a useful device is a small, hard bottle to which a sprayer (e.g., metered dose sprayer) is attached. In one embodiment, the dose is delivered by drawing the insulin solution into a chamber of defined volume, which chamber has an aperture dimensioned to aerosolize and aerosol formulation by forming a spray when a liquid in the chamber is compressed. The chamber is compressed to administer the insulin. In a specific embodiment, the chamber is a piston arrangement. Such devices are commercially available.

Alternatively, a plastic squeeze bottle with an aperture or opening dimensioned to aerosolize an aerosol formulation by forming a spray when squeezed. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the insulin.

Often, the aerosolization of a liquid or a dry powder formulation for inhalation into the lung requires a propellent. The propellent is any propellant generally used in the art. Specific nonlimiting examples of such useful propellants are a chloroflourocarbon, a hydrofluorocarbon, a hydochlorofluorocarbon, or a hydrocarbon, including trifluoromethane, dichlorodiflouromethane, dichlorotetrafluoroethanol, and 1,1,1,2-tetraflouroethane, or combinations thereof.

Systems of aerosol delivery, such as the pressurized metered dose inhaler and the dry powder inhaler are disclosed in Newman, S. P., Aerosols and the Lung, Clarke, S. W. and Davia, D. editors, pp. 197-22 and, in various aspects, are used in connection with the present disclosure.

Intracranial Formulations and Systems

In certain aspects, insulin is formulated in artificial cerebral spinal fluid (ACSF). Formulations for ACSF are known in the art. One exemplary formulation is made by formulating and mixing two electrolyte solutions, A and B. For solution A, NaCl (8.66 g), KCl (0.224 g), CaCl₂.2H₂O (0.206 g) and MgCl₂.6H₂O (0.163 g) are dissolved in 500 ml of pyrogen-free, sterile water. Solution B, in one aspect, is formulated by dissolving Na₂HPO₄.7H2O (0.214 g) and NaH₂PO₄.H₂O (0.027 g) 500 ml of pyrogen-free, sterile water. Solutions A and B are then mixed at 1:1 ratio to form ACSF having final ion concentrations of Na 150 mM, K 3.0 mM, Ca 1.4 mM, Mg 0.8 mM, P 1.0 mM and Cl 155 mM. Thus, the ACSF comprises ion concentrations similar to the known values for human CSF (Na 187.5 mM, K 2.6 mM, Ca 1.1 mM, Mg 1.1 mM, P 0.8 mM, Cl 119 mM and HCO₃ 23.3 mM). For further information on CSF and ACSF constituents see Dayson, H. Physiology of the Cerebrospinal Fluid, J. & A. Churchill, Ltd., London, 1967 and Biology Data Book, Volume III, 2nd ed., Fed. Am. Soc. Exper. Biol., Washington D.C., 1974, incorporated herein by reference.

### EXAMPLES

### Example 1

### Central Insulin Attenuates the Systemic Inflammatory Response.

Protocol Design

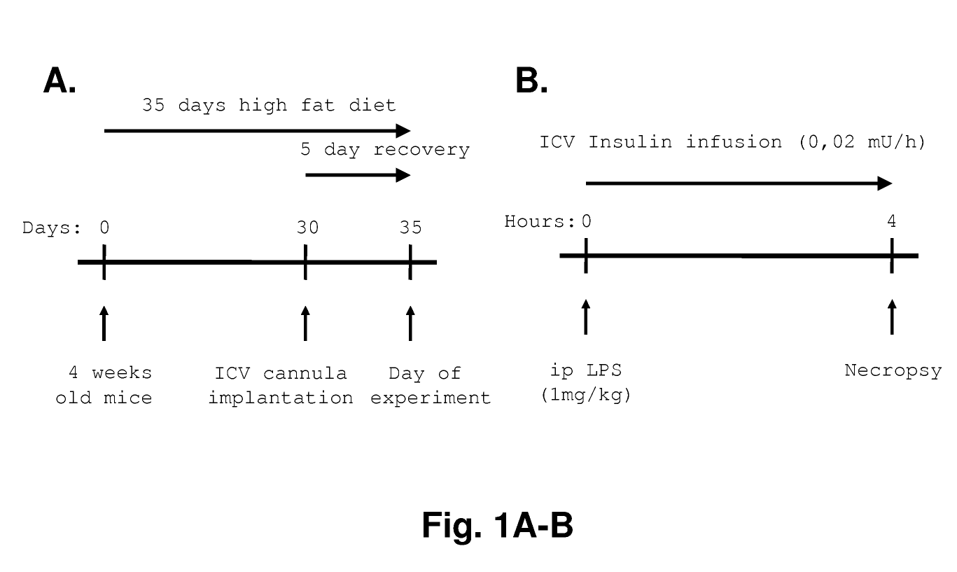

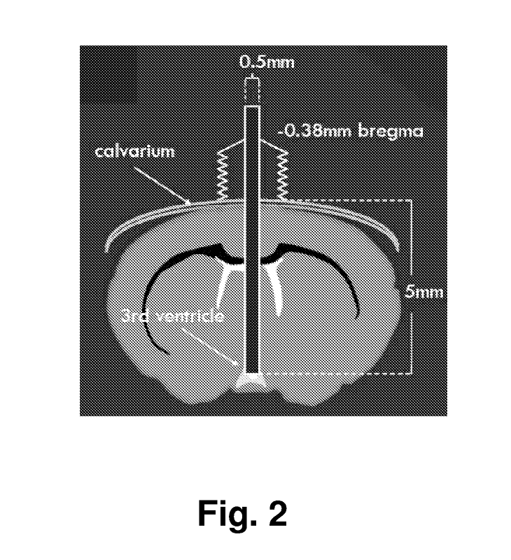

Male C57B1/6 mice were weaned at 4 weeks of age and fed a High Fat Diet (HFD) for 35 days to induce insulin resistance. After 30 days of HFD feeding, the mice underwent stereotactic surgery to implant intracerebroventricular (ICV) guide cannula (see, FIG. 2 for an illustration of cannula placement) followed by a 5-day recovery period until infusion experiments were carried out. On the day of the experiment, animals were placed in individual cages, received an intraperitoneal (i.p.) injection of E. coli (LPS) (1 mg/kg) and were continuously ICV infused using a micro infusion pump with Artificial CerebroSpinal Fluid (ACSF) or Insulin (0.005 mU/h) for 4 hours, yielding a total dose of 0.020 mU. See FIG. 1A-B for a graphical illustration of the protocol.

### Example 2

### Insulin Delivered to the CNS Decreases Circulating Pro-Inflammatory Cytokines

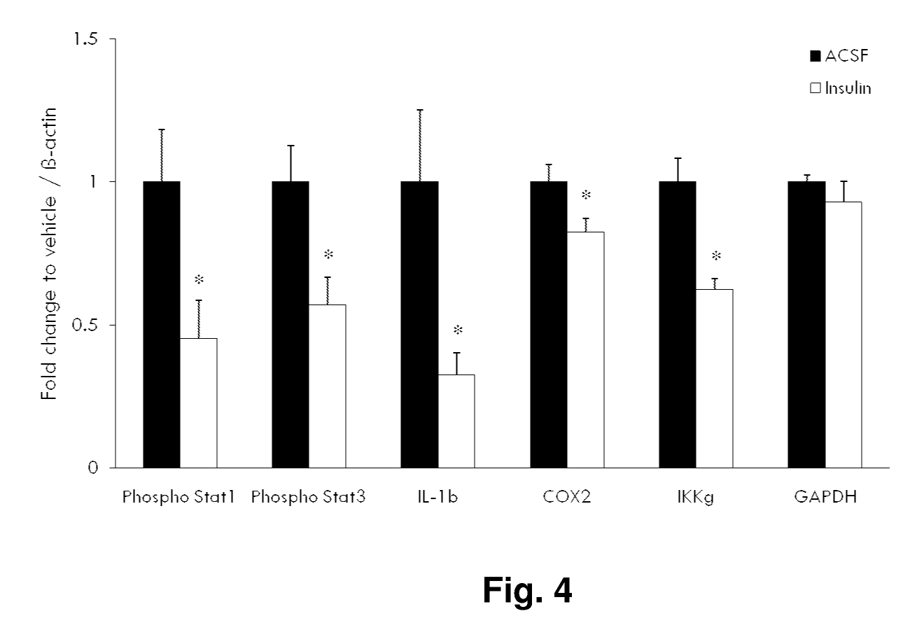

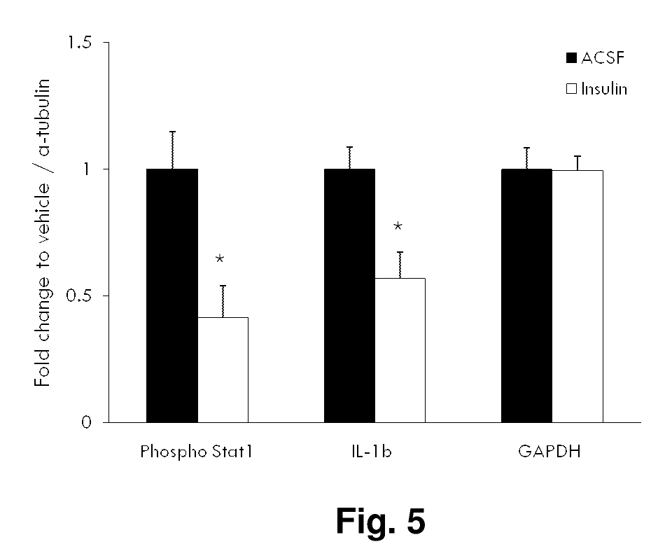

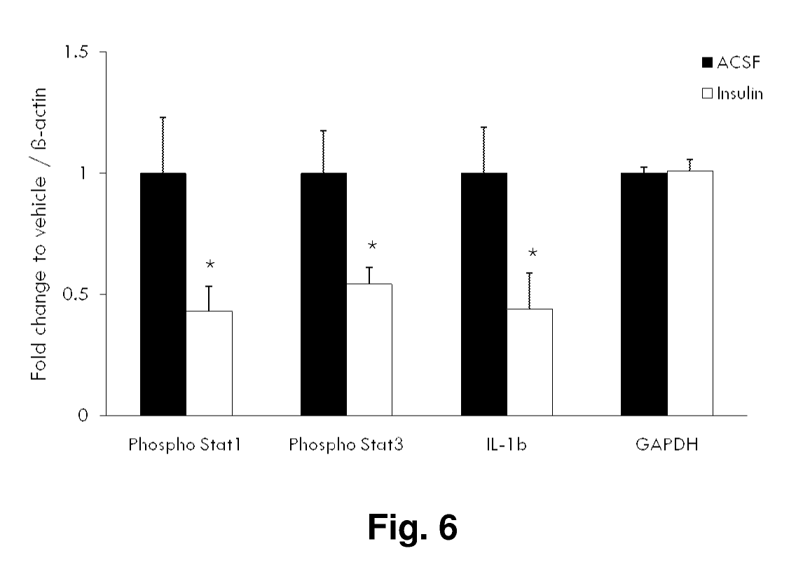

To investigate whether central insulin reduces cytokine signaling in peripheral tissues, the expression and phosphorylation of signal transducer and activator of transcription (Stat) was measured in lung (FIG. 4), spleen (FIG. 5) and liver (FIG. 6) tissue by western blot. After ICV insulin infusion, there was a marked inhibition of LPS induced phosphorylation of Stat1, which regulates the expression of many pro-inflammatory proteins such as COX2. Likewise, the activation of Stat3 which plays a central role in signaling of IL-6 and related cytokines was decreased in peripheral tissues after ICV insulin infusion. Lower protein levels of the pro-inflammatory cytokine IL-1β were detectable in the insulin-infused animals in liver, spleen and lung tissue. Lower IL-1β levels in lung tissue has been associated with improvement of acute lung injury, which is a clinically important sequelae in sepsis.

### Example 3

### The Effects of Glucose on Inflammation

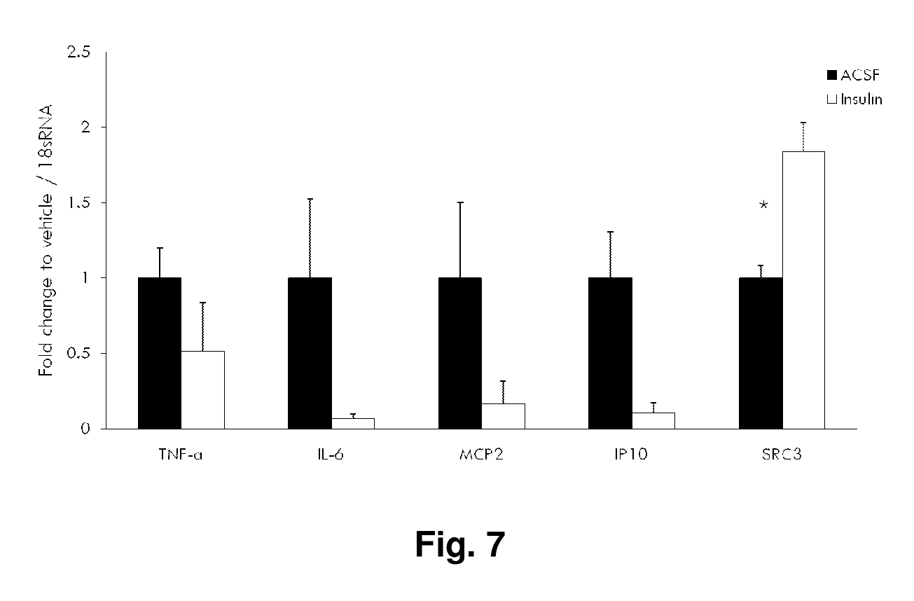

Because some of the metabolic effects of glucose are mediated via the hypothalamus, studies were undertaken to determine whether central hyperglycemia may potentate the inflammatory response to LPS. To this end, 4 mM glucose was centrally infused as depicted in FIG. 1. ICV infusion of glucose significantly increased Stat3 and Stat1 activation, and enhanced nuclear factor κB (NF-κB) signaling, represented through higher inhibitor κB kinase-β (IKK-β) levels. Central hyperglycemia also resulted in elevated protein disulfide isomerase (PDI) expression, an important marker of endoplasmatic reticulum (ER) stress (see, e.g., FIG. 7).

### Example 4

### ICV Insulin Improves Survival in a Mouse Model of Sepsis

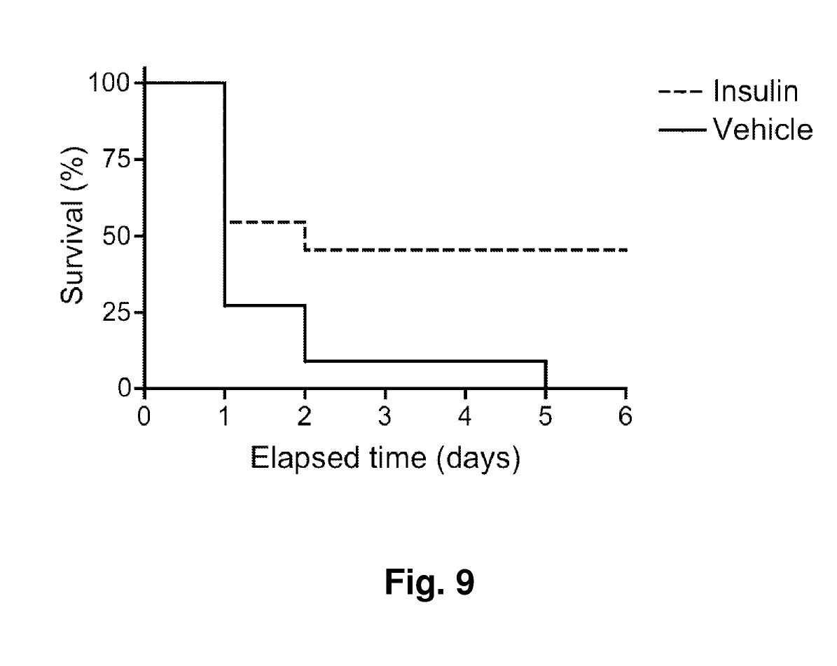

In view of the potent anti-inflammatory effects mediated by insulin when administered to the CNS, studies were undertaken to determine whether administration of insulin to the CNS would protect mice from death in a sepsis model. 4-week old C57B16 mice were fed high fat diet for 4 weeks and ICV cannulas were implanted as described above. Mice were injected with LPS (5 mg/kg, i.p.) and randomly assigned to receive ICV insulin or vehicle infusions for up to 5 days (n=11 per group). Survival was assessed daily and results were plotted, see, e.g., FIG. 9. As depicted in FIG. 9, ICV insulin significantly (p=0.0194) improved sepsis survival.

1. A method for treating systemic inflammation in a subject comprising administering insulin to the subject, wherein the insulin is delivered to the brain of the subject.

2. The method of claim 1, wherein the insulin is administered intracranially.

3. The method of claim 1, wherein the insulin is administered intranasally.

4. The method of claim 1, wherein administering insulin to the subject comprises administering a dose of insulin that does not result in hypoglycemia in the subject.

5. The method of claim 1, wherein administering insulin to the subject results in an insulin concentration in the brain that is higher than the serum insulin concentration in the subject.

6. (canceled)

7. (canceled)

8. The method of claim 1, wherein the insulin is administered in a solution.

9. The method of claim 1, wherein the insulin is administered intranasally by applying pressure to a carrier composition comprising the insulin.

10. The method of claim 1, wherein the insulin is administered by a syringe, a nebulizer, a respirator or a squeeze bottle.

11. The method of claim 1, wherein the subject is unconscious.

12. The method of claim 1, wherein the subject has systemic inflammatory response syndrome (SIRS).

13. The method of claim 1, wherein the subject has viral sepsis or bacterial sepsis.

14. (canceled)

15. The method of claim 13, wherein the bacterial sepsis is Salmonella, Staphylococcus, Streptococcus, Enterococcus, Escherichia, Klebsiella, Enterobacter, Pseudomonas, Yersinia, Campylobacter, Bacillus, Treponema or Fusobacterium sepsis.

16. The method of claim 1, further comprising administering an antibiotic to the subject.

17. (canceled)

18. The method of claim 1, further comprising administering to the subject a carrier which enhances insulin uptake in the brain.

19. The method of claim 18, the carrier that enhances insulin uptake in the brain is a liposome.

20. The method of claim 1, wherein the insulin is recombinant insulin is-fused to a peptide that enhances insulin uptake in the brain.

21. The method of claim 20, wherein the peptide that enhances insulin uptake in the brain is selected from the group consisting of TTQGNPQ (SEQ ID NO:1), YEQHHPG (SEQ ID NO:2), TTPHAWL (SEQ ID NO:3), TDNTAKN (SEQ ID NO:4), KIGFHGK (SEQ ID NO:5), KTHAQHE (SEQ ID NO:6), LSEQNRS (SEQ ID NO:7), PMPRPSS (SEQ ID NO:8), SLTTSTL (SEQ ID NO:9) and ATTKFSG (SEQ ID NO:10).

22. The method of claim 21, wherein the peptide that enhances insulin uptake in the brain is TTPHAWL (SEQ ID NO:3).

23. A composition comprising insulin at a dosage effective to treat systemic inflammation.

24. (canceled)

25. (canceled)

26. The composition of claim 23, further comprising a carrier that enhances insulin uptake in the brain.

27. The composition of claim 26, wherein the carrier is a liposome.

28. (canceled)

29. The composition of claim 28, wherein the insulin is recombinant insulin fused to a peptide that enhances insulin uptake in the brain.

30. The composition of claim 29, wherein the peptide that enhances insulin uptake in the brain is selected from the group consisting of TTQGNPQ (SEQ ID NO:1), YEQHHPG (SEQ ID NO:2), TTPHAWL (SEQ ID NO:3), TDNTAKN (SEQ ID NO:4), KIGFHGK (SEQ ID NO:5), KTHAQHE (SEQ ID NO:6), LSEQNRS (SEQ ID NO:7), PMPRPSS (SEQ ID NO:8), SLTTSTL (SEQ ID NO:9) and ATTKFSG (SEQ ID NO:10).

31. The composition of claim 23, wherein the composition is administered by a syringe, a nebulizer, a respirator or a squeeze bottle.

32. The composition of 30 claim 23, further comprising one or more antibiotics.

33. A polypeptide comprising human insulin peptide fused to a targeting peptide wherein the targeting peptide enhances insulin uptake to the brain when the polypeptide is administered to a subject intranasally.

34. The polypeptide of claim 33, wherein the targeting peptide comprises the sequence TTQGNPQ (SEQ ID NO:1), YEQHHPG (SEQ ID NO:2), TTPHAWL (SEQ ID NO:3), TDNTAKN (SEQ ID NO:4), KIGFHGK (SEQ ID NO:5), KTHAQHE (SEQ ID NO:6), LSEQNRS (SEQ ID NO:7), PMPRPSS (SEQ ID NO:8), SLTTSTL (SEQ ID NO:9) and ATTKFSG (SEQ ID NO:10).

35. The polypeptide of claim 34, wherein the targeting peptide comprises the sequence TTPHAWL (SEQ ID NO:3).

36. The polypeptide of claim 33, wherein the targeting peptide is fused to the amino terminus of the insulin peptide.

37. The composition according to claim 23 further comprising insulin formulated in artificial cerebrospinal fluid (ACSF).

38. A kit for the treatment of sepsis comprising one or more doses of insulin formulated for intranasal administration and one or more antibiotics, wherein the one or more doses of insulin are effective to treat sepsis.

39. An insulin delivery system comprising insulin formulated for intranasal delivery and a pressure source sufficient to deliver the insulin to the intranasal passage of a subject.

40. The insulin delivery system of claim 39, wherein the pressure source is sufficient to deliver the insulin to the intranasal passage of an unconscious subject.

41. (canceled)

42. An insulin delivery system comprising insulin formulated for intracranial delivery and a brain infusion cannula.

43. The composition according to claim 23 wherein the dosage is effective to treat systemic inflammation when administered intranasally, said composition formulated for intranasal delivery.
